(19) United States
(12) Patent Application Publication
(10) Pub. No.: US 2020/0038348 A1
(43) Pub. Date: Feb. 6, 2020

(54) METHODS AND MATERIALS FOR REDUCING SCAR FORMATION DURING WOUND HEALING

(71) Applicant: Mayo Foundation for Medical Education and Research, Rochester, MN (US)

(72) Inventors: Alexander Meves, Rochester, MN (US); Saranya P. Wyles, Rochester, MN (US); Steven L. Moran, Rochester, MN (US)

(73) Assignee: Mayo Foundation for Medical Education and Research, Rochester, MN (US)

(21) Appl. No.: 16/485,199

(22) PCT Filed: Feb. 16, 2018
(86) PCT No.: PCT/US2018/000052

§ 371 Date: Aug. 12, 2019

Related U.S. Application Data
(60) Provisional application No. 62/460,563, filed on Feb. 17, 2017.

(51) Int. Cl. A61K 31/155; A61P 17/02

(52) U.S. Cl. CPC A61K 31/155; A61P 17/02

(57) ABSTRACT

This document provides methods and materials for reducing scar formation (e.g., scar formation during wound healing) and/or for reducing keloid formation (e.g., keloid formation that occurs during wound healing or keloid formation that does not occur during wound healing). For example, methods and materials for topically administering pentamidine to reduce scar formation and/or to reduce keloid formation are provided.

### CROSS-REFERENCE TO RELATED APPLICATIONS

This application claims priority to U.S. Application Ser. No. 62/460,563, filed on Feb. 17, 2017. The disclosure of the prior application is considered part of the disclosure of this application, and is incorporated in its entirety into this application.

### BACKGROUND

### 1. Technical Field

This document relates to methods and materials for reducing scar formation and/or for reducing keloid formation. For example, this document relates to methods and materials for topically administering pentamidine (4,4′-[pentane-1,5-diylbis(oxy)]dibenzenecarboximidamide) to reduce scar formation and/or to reduce keloid formation.

### 2. Background Information

Hypertrophic scars and keloids are characterized by excessive fibrosis and extracellular matrix (ECM) deposition and can be functionally and cosmetically problematic. Treatment of hypertrophic scars and keloids can be challenging.

Inflammatory cells and cytokines involved in excessive inflammation during wound healing facilitate fibroblast proliferation and collagen deposition, leading to pathologic scar formation. Indeed, skin wound healing is a programmed process that includes inflammation, proliferation, maturation, and reshaping. The inflammatory response functions as an anti-infection immune barrier and can stimulate collagen synthesis to repair the wound. Therefore, moderate inflammation is advantageous for wound healing.

### SUMMARY

This document provides methods and materials for reducing scar formation (e.g., scar formation during wound healing) and/or for reducing keloid formation (e.g., keloid formation that occurs during wound healing or keloid formation that does not occur during wound healing). For example, this document provides methods and materials for topically administering pentamidine to reduce scar formation and/or to reduce keloid formation.

As described herein, a composition containing pentamidine (e.g., a composition containing pentamidine as the sole active ingredient) can be topically administered to the skin of a mammal (e.g., a human) to reduce scar formation and/or to reduce keloid formation. For example, a skin care composition containing pentamidine can be topically administered to a wound (e.g., an accidental cut or surgical incision) in a manner that results in wound healing with reduced scar formation as compared to the wound healing that occurs when a comparable wound heals without the topical administration of a skin care composition containing pentamidine. Having the ability to use the skin care compositions provided herein in a manner that allows a user (e.g., a human) to experience wound healing with reduced scar formation can allow the user to achieve a desired skin healing without undesirable scar formation.

In general, one aspect of this document features a method for healing a wound of a mammal with reduced scar formation. The method comprises, or consists essentially of, topically applying a skin care composition comprising pentamidine (or a salt thereof) or pentamidine isethionate (or a salt thereof) to the wound of the mammal, wherein the wound heals with less observable scaring than that which is observable from a comparable wound healed in the absence of the composition. The mammal can be a human. The wound can be a surgical incision. The wound can be an accidental wound. The skin care composition can comprise from about 0.01 percent to about 10 percent, by weight, of the pentamidine (or a salt thereof) or pentamidine isethionate (or a salt thereof). The wound can heal with less observable hypertrophic scaring than that which is observable from a comparable wound healed in the absence of the composition. The skin care composition can be applied topically to the wound at least daily for about 5-45 days, about 10-45 days, or at least 30 days.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIGS. 1A-D. Pentamidine inhibits SPP1 expression. (A) Schematic representation of the DuoGlo construct used for high throughput compound screening. (B-C) SPP1 promoter activity after exposure of WM858 Dual-Glo cells to Vitamin D (B) or retinoid (C). Mean±s.d.; n=3; *P<0.05. (D) Dual-Glo-tagged SPP1 promoter was screened against a library of pharmaceutically active compounds (LOPAC).

FIG. 2 contains diagrams of surgical ligation of leporine auricular blood vessels.

FIG. 3 contains photographs of an ischemic rabbit ear model.

FIG. 4 contains the chemical structure of pentamidine isethionate (Pentam 300).

FIG. 5 contains a table listing the indicated treatment protocols.

FIG. 6 contains photographs of untreated hypertrophic scaring.

FIG. 7 contains photographs of hypertrophic scaring after topical treatment with only a base compound (Pracasil™-Plus).

FIG. 8 contains photographs of hypertrophic scaring after topical treatment with 2 percent pentamidine in a base compound (Pracasil™-Plus). Minimal hypertrophic scaring was observed as compared to the level of hypertrophic scaring observed in FIGS. 6 and 7.

FIG. 9 contains photographs of the effects of osteopontin inhibition in the epidermis. p16 expression levels in rabbit skin with (+) and without (−) pentamidine treatment are shown. Osteopontin inhibition decreased p16 levels in dermal and epidermal regions (n=6 per group).

FIG. 10 contains photographs of the dose-dependent effect of osteopontin inhibition with pentamidine. Small doses (0.5% and 1%) had no response to minimal reduction in hypertrophic scar formation. At 5% drug dose, pentamidine was highly concentrated in the base, resulting in non-healing wound and abscess formation. Effective re-epithelization was occurred when 2% pentamidine was used.

### DETAILED DESCRIPTION

This document provides methods and materials involved in treating a mammal's skin to reduce scar formation and/or to reduce keloid formation by topically administering pentamidine (or a salt thereof) or pentamidine isethionate (or a salt thereof) to the mammal. Any appropriate mammal can be treated as described herein. For example, humans and other primates such as monkeys can be treated with pentamidine (or a salt thereof, pentamidine isethionate, or a salt thereof) to reduce scar formation and/or to reduce keloid formation. In some cases, dogs, cats, horses, bovine species, porcine species, mice, or rats can be treated with pentamidine (or a salt thereof, pentamidine isethionate, or a salt thereof) as described herein to reduce scar formation and/or to reduce keloid formation. In addition, a mammal having any particular type of wound can be treated as described herein. For example, a mammal having an accidental wound or a surgical incision can be treated with pentamidine (or a salt thereof, pentamidine isethionate, or a salt thereof) as described herein to reduce scar formation and/or to reduce keloid formation. In some cases, a mammal (e.g., a human) developing or expected to develop keloids can be treated with pentamidine (or a salt thereof, pentamidine isethionate, or a salt thereof) as described herein to reduce keloid formation.

Once identified as having a wound or a susceptibility to develop keloid(s), the mammal can be topically administered a skin care composition containing pentamidine (or a salt thereof, pentamidine isethionate, or a salt thereof) to the affected areas of skin. In some cases, pentamidine (or a salt thereof, pentamidine isethionate, or a salt thereof) can be topically administered as the sole active ingredient. In other cases, pentamidine (or a salt thereof; pentamidine isethionate, or a salt thereof) can be topically administered in combination with corticosteroids, niclosamide, 5-fluorouracil, a hydrogel scaffold, or combinations thereof. Examples of corticosteroids that can be used in combination with pentamidine include, without limitation, hydrocortisone and triamcinolone.

A skin care composition containing pentamidine (or a salt thereof, pentamidine isethionate, or a salt thereof) can be repeatedly applied to a mammal's skin (e.g., a human's skin) for any appropriate period of time (e.g., days, weeks, or months). For example, a skin care composition provided herein can be applied at least once a day (e.g., once or twice daily) or at least once a week (e.g., 1, 2, 3, 4, or 5 times a week) for at least 1, 2, 3, 4, 5, 6, 7, or 8 weeks or for at least 1, 2, 3, 4, 5, 6, 7, or 8 months. In some cases, a skin care composition containing pentamidine (or a salt thereof, pentamidine isethionate, or a salt thereof) can be repeatedly applied to a mammal's skin (e.g., a human's skin) until the wound being treated is healed.

A skin care composition provided herein can contain any appropriate amount of pentamidine (or a salt thereof, pentamidine isethionate, or a salt thereof). For example, a skin care composition provided herein can contain from about 0.01 percent to about 15 percent (e.g., from about 0.01 percent to about 10 percent, from about 0.01 percent to about 5 percent, from about 0.1 percent to about 15 percent, from about 1 percent to about 15 percent, from about 1 percent to about 5 percent, from about 0.01 percent to about 0.1 percent, from about 0.1 percent to about 1 percent, or from about 1 percent to about 10 percent), by weight, of pentamidine (or a salt thereof; pentamidine isethionate, or a salt thereof). In some cases, a skin care composition provided herein can include a fixed number of active ingredients. For example, a skin care composition provided herein can be formulated to have no more than one (i.e., pentamidine, a salt thereof, pentamidine isethionate, or a salt thereof), two, three, or four active ingredients. In some cases, a skin care composition provided herein can be formulated to have two active ingredients (e.g., pentamidine and niclosamide) and no other active ingredients.

A skin care composition provided herein can be in any appropriate form for topical application to skin (e.g., human skin). For example, a skin care composition provided herein can be in the form of a cream, gel, spray (e.g., an aerosol spray or non-aerosol spray), ointment, lotion, foam, solution, paste, or clay.

In some cases, a skin care composition provided herein can contain one or more optional classes of ingredients such pH adjusters, preservatives, solvents, viscosity increasing agents, excipients, emulsifiers, and emollients. In some cases, a skin care composition provided herein can contain topical silicone-based compounding base agent such as Pracasil™-Plus.

The final pH of the undiluted skin care composition provided herein can be between about 5 and about 8. To obtain such a final pH, the pH of the composition can be adjusted. A pH-adjusting agent can be used to adjust the pH. The pH adjustment can be accomplished with any of a wide variety of acids. Examples of acids that can be used include, without limitation, citric acid, acetic acid, benzoic acid, glycolic acid, lactic acid, malic acid, and sulfuric acid should the composition have a pH too high (e.g., greater than 8 before adjustment). Likewise, the pH adjustment can be accomplished with any of a wide variety of bases should the composition have a pH too low (e.g., less than 5 before adjustment). Examples of bases that can be used to lower the pH of these formulations can be potassium hydroxide, potassium carbonate, sodium carbonate, sodium hydroxide, ethanolamine, or triethanolamine.

In some cases, to preserve a skin care composition provided herein and prevent microbial growth, a preservative can be included in a skin care composition provided herein. Examples of preservatives that can be included in a skin care composition provided herein include, without limitation, butylated hydroxytoluerie, benzoic acid, benzyl alcohol, butylparaben, propylparaben, methyparaben, DMDM hydantoin, potassium benzoate, methylisothiazolinone, methylchloroisothiazolinone, phenoxyethanol, quaterium-8, quaterium-14, quaterium-15, triclosan, zinc pyrithione, and zinc salicylate.

In some cases, one or more solvents can be included in a skin care composition provided herein as an optional ingredient. Examples of solvents that can be included in a skin care composition provided herein include, without limitation, butanediol, isoparaffin, cyclomethicone, ethoxyglycol, glycerin, mineral oil, polydimethlysiloxanes, propylene glycol, and propanediol.

In some cases, to help acquire a desired finished product thickness or viscosity, one or more viscosity modifiers can be included in a skin care composition provided herein. Examples of viscosity modifiers that can be included in a skin care composition provided herein include, without limitation, zinc oxide, ammonium xylene sulfonate, bentonite, calcium alginate, cocamide DEA, cocamide MEA, dextrin, hectorite, ethylcellulose, guar hydroxypropyltrimonium chloride, hydroxypropyl guar, hydrated silica, lauramide DEA, lauramide MEA, magnesium chloride, methylcellulose, pectin, polyethyleneglycol (PEGs), sodium chloride, sodium stearate, xanthan gum, and zea mays (corn starch).

In some cases, one or more excipients can be included in a skin care composition provided herein. Examples of excipients that can be included in a skin care composition provided herein include, without limitation, menthol, diglyceride, triglyceride, stabilizing agents, antioxidants, fragrances, and colorants.

In some cases, one or more emulsifiers and emollients can be included in a skin care composition provided herein. Examples of emulsifiers and emollients that can be included in a skin care composition provided herein include, without limitation, ceteareth-20, cetostearyl alcohol, diethylaminethyl stearate, glyceryl dilaurate, glyceryl monostearate, glyceryl stearate, PEG-100 stearate, octyldodecyl stearoyl stearate, polysorbate 80, quaternium-2β, stearyl alcohol, sodium PCA, dimethicone, cyclomethicone, propylene glycol, and polysiloxane derivatives. In some cases, an emulsifier or emollient can be present in a skin care composition provided herein at an amount from about 20 percent to about 80 percent, by weight.

The invention will be further described in the following examples, which do not limit the scope of the invention described in the claims.

### EXAMPLES

### Example 1

### Wound Healing with Reduced Scar Formation

Cytokines such as osteopontin (SPP1) that are produced by inflammatory and other cell types during wound healing can promote excessive fibroblast proliferation and the synthesis of excessive extracellular matrix (ECM). These events can result in abnormal ECM composition and ultimately hypertrophic scar formation. The following was performed to identify and characterize anti-aging drugs that can inhibit pro-inflammatory cytokine production in wound healing responses and that can be used to optimize the regenerative microenvironment during wound healing.

Although the incidence rates of hypertrophic scarring vary from 40% to 70% following surgery to up to 91% following burn injury, depending on the depth of the wound (Deitch et al., J. Trauma, 23:895-8 (1983); and Lewis et al., Burns, 16:176-8 (1990)), there is currently no effective topical treatment regimen for hypertrophic scar formation. Many treatments have been tried, with limited success, to reduce abnormal scarring (Friedstat et al., Ann. Plast. Surg., 72:S198 (2014)). Unfortunately, recurrence rates are high, and multiple or combination therapies are often required to reduce the scar volume and achieve functional and/or cosmetic improvement (Tredget et al., Surg. Clin. North Am., 94:793 (2014)).

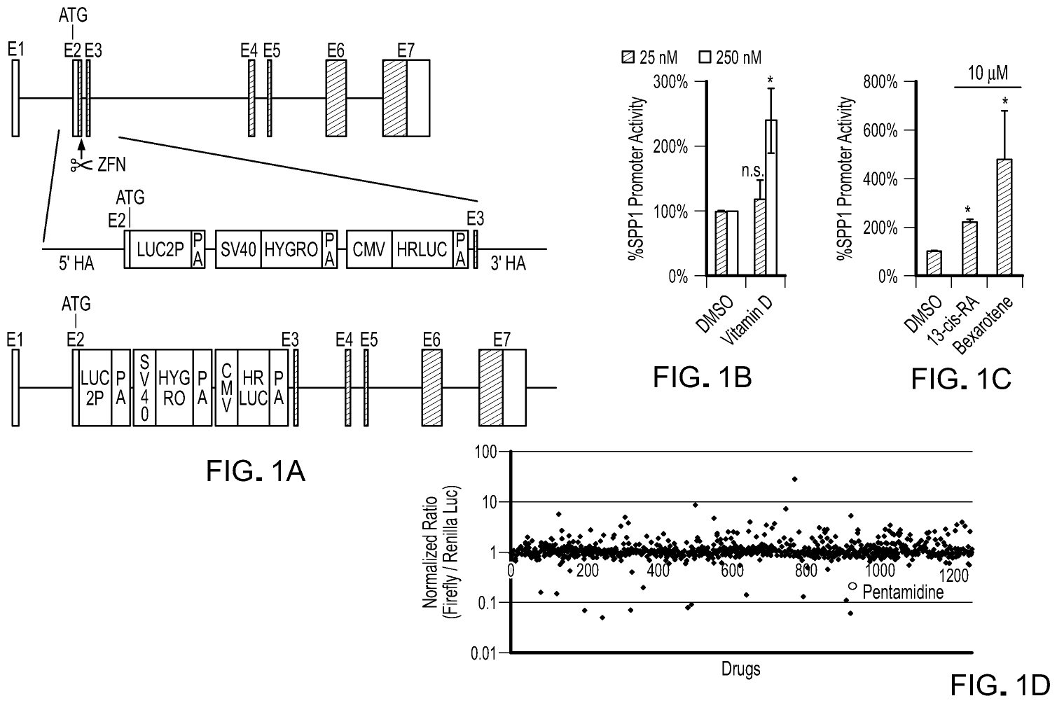

SPP1 is a pro-inflammatory inducer of skin fibrosis and skin aging. It was hypothesized that pharmaceutically active compounds that silence SPP1 expression in cells and tissues can be used to treat skin inflammation and scarring. To screen for transcriptional silencers of SPP1 expression, cells with high endogenous SPP1 expression (e.g., melanoma cells) were identified. Then, the endogenous SPP1 promoter was tagged in melanoma cells with a dual luciferase reporter to create Dual-Glo cells using a genome editing approach. Firefly luciferase was introduced to assay SPP1 promoter activity; renilla luciferase was coupled to the CMV promoter as a loading control (FIG. 1A). SPP1 promoter was activated by Vitamin D as it was shown to harbor vitamin D response elements (FIG. 1B). Retinoic acid receptor agonists, reported previously to induce SPP1, also induced SPP1 promoter activity in Dual-Glo cells (FIG. 1C). When an unbiased screen of pharmaceutically active compounds was performed to identify compounds that inhibit SPP1 promoter activity, pentamidine was identified. In fact, pentamidine was found to inhibit SPP1 promoter activity by greater than about 80 percent (FIG. 1D) with minimal in vitro cell toxicity.

Pentamidine isethionate (a diamidino compound), which is used in the treatment of Pneumocystis carinii pneumonia, was primarily studied in its aerosolized and oral form (Sands et al., Rev. Infect. Dfs., 7:625 (1985); and Rosenthal et al., Toxtcol. Appl. PhQrmQCOL, 107:555 (1990)). It was postulated to inhibit the release of inflammatory mediators from alveolar macrophages, which may be associated with its anti-parasite activity. Another study examined topical pentamidine (at minimum dose, 20 μg) in the inhibition of contact hypersensitivity reaction in Langerhans cells

(Blaylock et al., J. Immunol., 147(7):2116-21 (1991)).

The following was performed to investigate the use of topical pentamidine for reducing scar formation (e.g., hypertrophic scar formation). It was hypothesized that the application of pentamidine as a topical agent in a base compound such as Pracasil™-Plus would inhibit tissue fibrosis and promote optimal tissue regeneration during wound healing. To test this hypothesis, the effects of topical pentamidine gel was studied in an established in vivo model of hypertrophic scar formation (i.e., the ischemic rabbit ear model). Topical pentamidine gel application significantly inhibited hypertrophic scar formation in this model.

### Hypertrophic Scar Formation Model

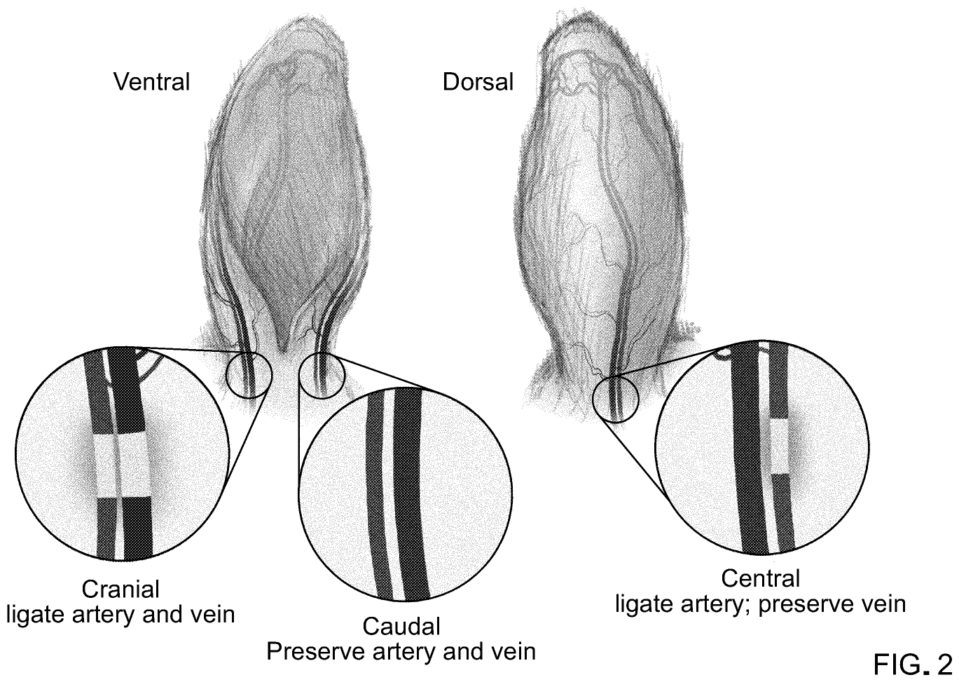

Topical pentamidine was evaluated using a hypertrophic scar formation rabbit ear model. Briefly, surgical ligation of leporine auricular vessels induced an ischemic landscape for hypertrophic scar production. Specifically, a cranial artery and vein were ligated; the caudal artery and vein were preserved; and the central artery was ligated, while the central vein was preserved in the rabbit ear (FIG. 2).

All surgeries were conducted in a disinfected area designated for surgical procedures and equipped with necessary lighting and equipment. Briefly, rabbits received intramuscular buprenorphine and enrofloxicin (5-10 mg/kg) prior to the surgery. Anesthesia was induced by a mixture of ketamine and intramuscular xylazine and monitored by medical sciences support staff. Study staff performed surgeries using sterile instruments, drapes, gloves, and gown. Following vascular ligation, one linear full-thickness skin wound was created on the ventral side of each ear with a surgical blade. The skin and perichondrium were removed. The base on which granulation and epithelialization take place was the cartilage. The edges of the wound were closed with 4-0 Nylon sutures.

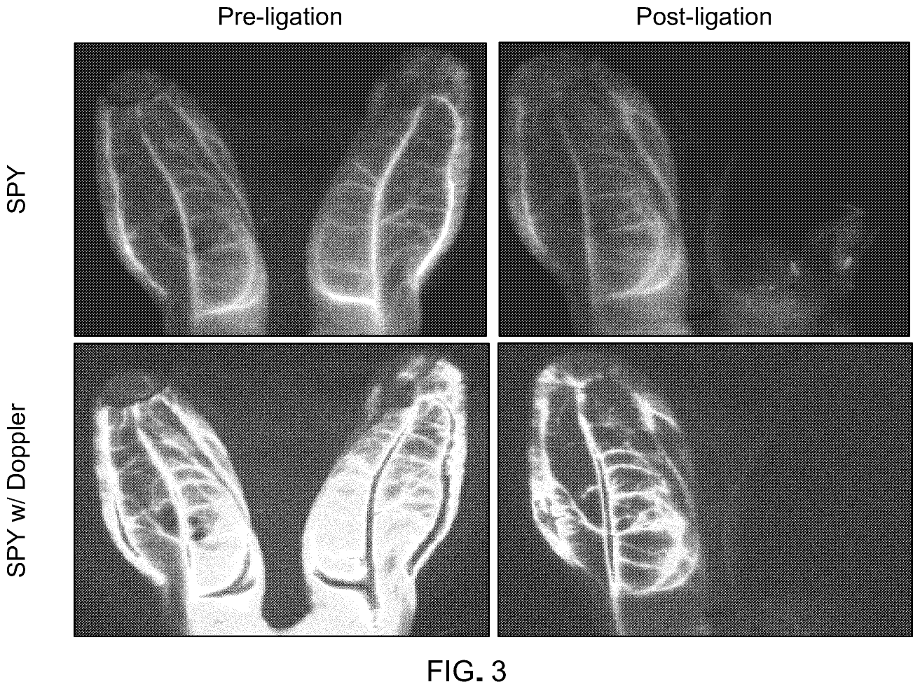

Following ischemic wounding, the relative ischemia created in the ear at time of surgery and time of sacrifice was assessed with a fluorescent light assisted angiography (Spy Elite, LifeCell). Perfusion of the ear was assessed using the Spy Elite system following intravenous administration of indocyanine green, which utilizes infrared light to detect the dye (FIG. 3).

### Topical Gel Development

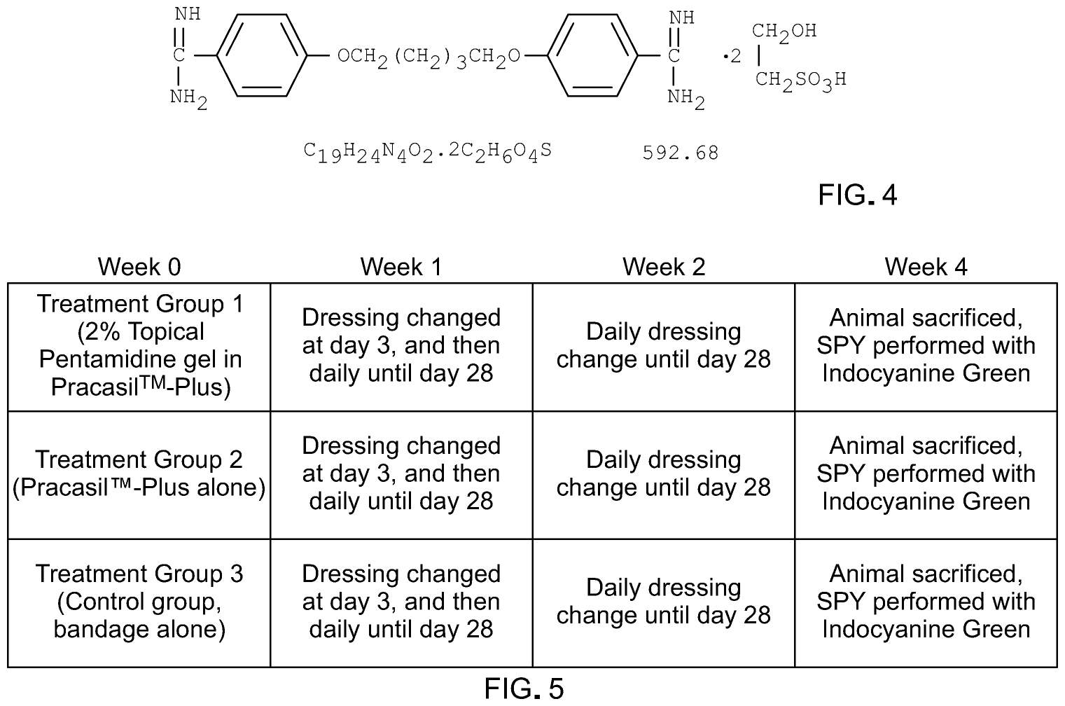

Pentam 300 (pentamidine isethionate) is an anti-protozoal agent and is a sterile, nonpyrogenic, lyophilized product. Pentamidine isethionate is a white crystalline powder soluble in water and glycerin, soluble in alcohol, and insoluble in ether, acetone, and chloroform. It is chemically designated as 4,4-[1,5-pentanediylbis(oxy)]bis-benzenecarboximidamid (FIG. 4). Professional Compounding Centers of America (PCCA) Pracasil™-Plus is a topical silicone-based compounding base agent created for scar therapy. Pracasil formulation ingredients include cyclopentasiloxane, polysilicone-11, PEG-16 macadamia glycerides, dimethicone, C30-45 alkyl cetearyl dimethicone crosspolymer, pentaclethra macroloba seed oil, oenocarpus bataua pulp oil, phosphatidylcholine, tocopheryl acetate, and BHT.

30 g of 2% topical gel pentamidine in PCCA Pracasil™-Plus was produced for the initial animal studies in an USP-grade facility. This gel was stored at room temperature and was stable until the end of the study period (4 weeks).

### Study Design

Following vascular ligation and linear wounding, a sterile occlusive dressing (TegaDerm, 3M) was used to cover the wound site. Animals were allowed to recover for three days without wound disturbance. After 72 hours, the dressing was changed, and the treatment was initiated. Rabbits were assigned to treatment groups: (1) 2% topical pentamidine gel in Pracasil™-Plus, (2) Pracasil™-Plus alone, or (3) control group with synthetic bandage alone (FIG. 5). Daily dressing changes occurred until day 28. About 1/8 teaspoon (1 oz) of the treatment agent was applied with each dressing change. Rabbits were trained on the animal snuggler for ease during treatment application.

Animals were monitored daily for any signs of postoperative discomfort, wound infection, weight loss, loss of appetite, and loss of contracture fixation. Any complications such as cage sores, wound infections, weight loss, or wound dehiscence were reviewed with the veterinarian and managed in a way designed to minimize discomfort or suffering. Any complication that could not be resolved resulted in the animal being removed from the study. Six rabbits were used in this study. For three rabbits, the left ear received Treatment Group 1 (n=3), and the right ear received Treatment Group 2 (n=3). The remaining three rabbits did not receive treatment (Treatment Group 3). One rabbit was excluded from the study due to wound infection:

### Results

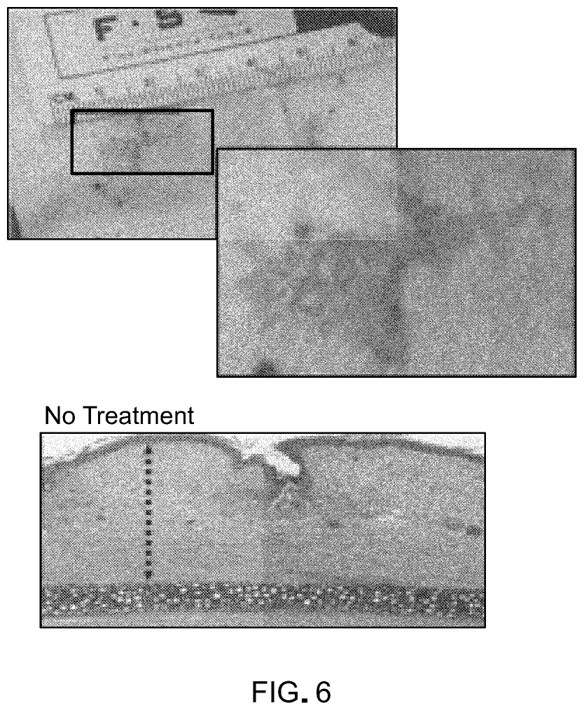

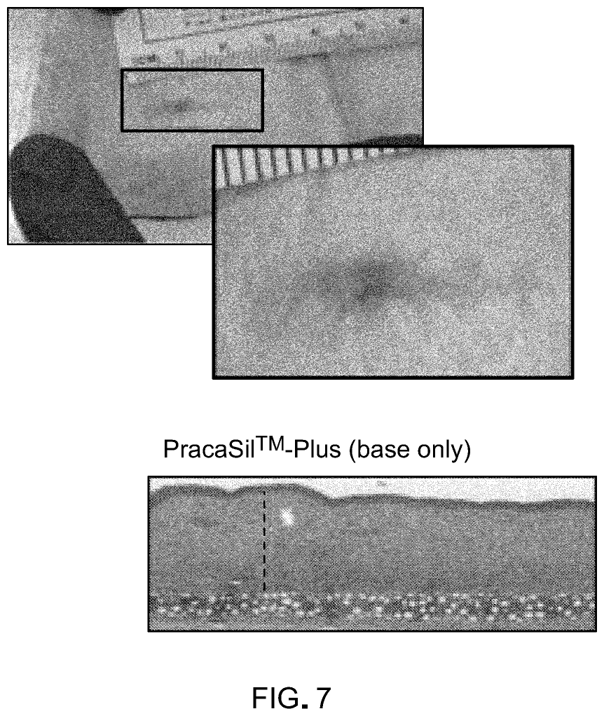

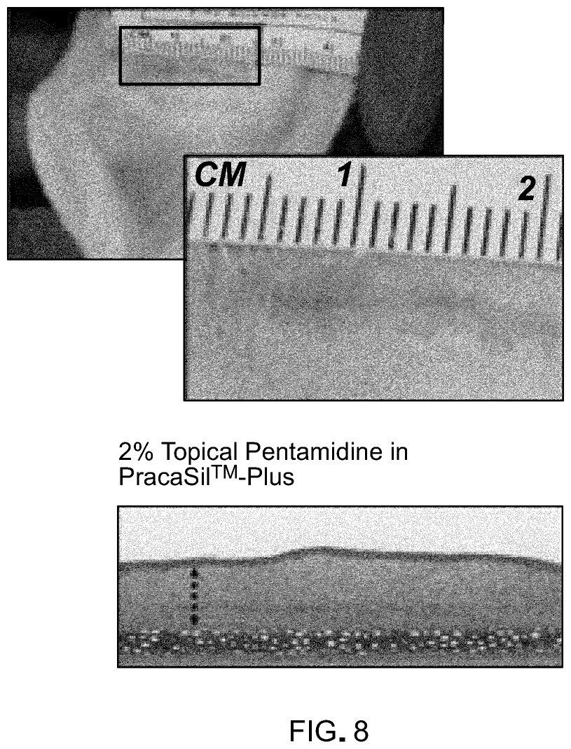

Without pharmacological management, the animals of Treatment Group 3 exhibited hypertrophic scar formation (FIG. 6). The ears receiving the treatment of Treatment Group 1 exhibited observable scar reduction following four weeks of topical gel application with 2% pentamidine in Pracasil™-Plus base (FIG. 8). Comparatively, the ears receiving the treatment of Treatment Group 2 exhibited some observable scar reduction, but to a lesser degree than that observed for the Treatment Group 1 ears (FIG. 7).

These results demonstrate that pentamidine can be used topically to reduce hypertrophic scar formation.

### Example 2

### Wound Healing with Reduced Scar Formation

Female New Zealand white rabbit ears were used. In total, twelve linear wounds were created using minimally invasive vascular ligation in auricular blood vessels to create ischemic hypertrophic scars. Following surgical ligation, linear wounds were treated with either osteopontin inhibitor 2% topical pentamidine (n=6; left ear) or base only (n=6; right ear) for four weeks. After the treatment period, samples were fixed in 10% formaldehyde, embedded in paraffin and stained for p16 expression.

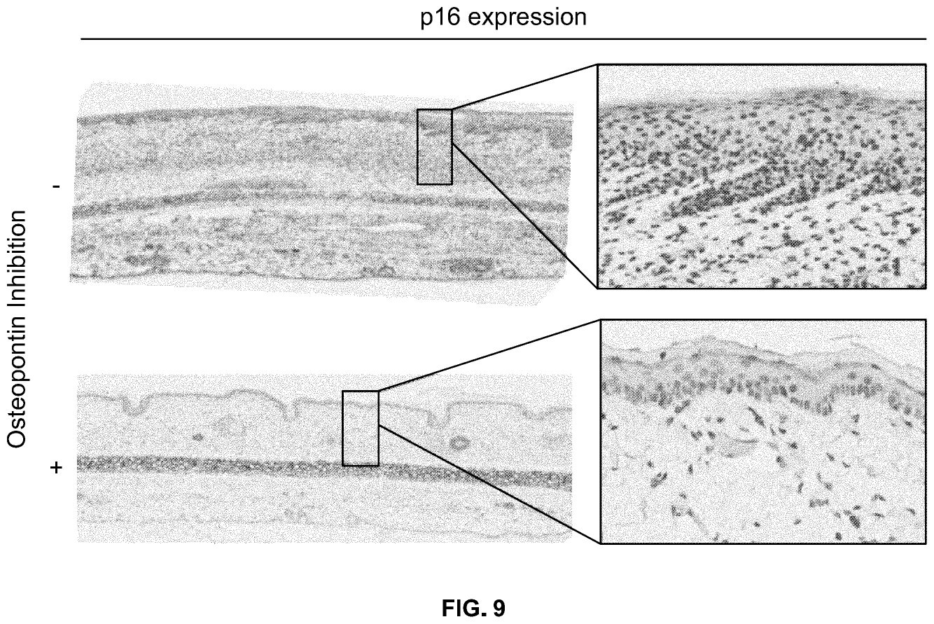

Osteopontin inhibitor using 2% topical pentamidine reduced p16 expression levels in dermal and epidermal regions (FIG. 9).

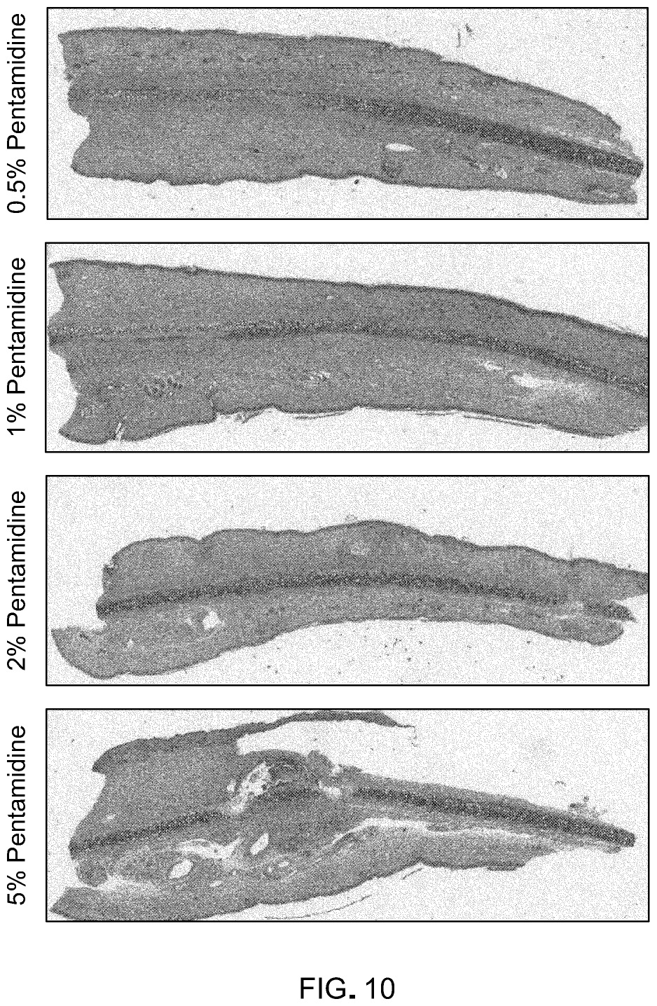

Female New Zealand white rabbits ears were used. In total, twelve linear wounds were created using minimally invasive vascular ligation in auricular blood vessels to create ischemic hypertrophic scars. Following surgical ligation, linear wounds were treated with the following dose titration: 0.5% pentamidine (n=3); 1% pentamidine (n=3); 2% pentamidine (n=3); or 5% pentamidine (n=3). After the treatment period, samples were fixed in 10% formaldehyde, embedded in paraffin, and stained with H&E and Masson's trichrome. Small doses (0.5% and 1%) had no response to minimal reduction in hypertrophic scar formation (FIG. 10). At 5% drug dose, pentamidine was highly concentrated in the base, resulting in non-healing wound and abscess formation (FIG. 10). Effective re-epithelization was occurred when 2% pentamidine was used (FIG. 10).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for healing a wound of a mammal with reduced scar formation, wherein said method comprises topically applying a skin care composition comprising pentamidine or pentamidine isethionate to said wound of said mammal, wherein said wound heals with less observable scaring than that which is observable from a comparable wound healed in the absence of said composition.

2. The method of claim 1, wherein said mammal is a human.

3. The method of claim 1, wherein said wound is a surgical incision.

4. The method of claim 1, wherein said wound is an accidental wound.

5. The method of claim 1, wherein said skin care composition comprises from about 0.01 percent to about 10 percent, by weight, of said pentamidine or pentamidine isethionate.

6. The method of claim 1, wherein said wound heals with less observable hypertrophic scaring than that which is observable from a comparable wound healed in the absence of said composition.

7. The method of claim 1, wherein said skin care composition is applied topically to said wound at least daily for at least 30 days.
